(19) United States
(12) Patent Application Publication
(10) Pub. No.: US 2018/0360951 A1
(43) Pub. Date: Dec. 20, 2018

(54) METHOD, COMPOSITION & APPARATUS FOR THE TREATMENT OF TYPE I ALLERGIC HYPERSENSITIVITIES BY IMMUNE DESENSITIZATION THROUGH SLIT IMMUNOTHERAPY WITH UNADULTERATED ALLERGEN, VITAMIN A AND VITAMIN D

(71) Applicant: EVROS VASSILIOU, FRANKLIN LAKES, NJ (US)

(72) Inventor: EVROS VASSILIOU, FRANKLIN LAKES, NJ (US)

(21) Appl. No.: 15/908,672

(22) Filed: Feb. 28, 2018

Related U.S. Application Data
(60) Provisional application No. 62/522,256, filed on Jun. 20, 2017.

(51) Int. Cl. A61K 39/36; A61K 31/07; A61P 37/08

(52) U.S. Cl. CPC A61K 39/36; A61K 2039/542; A61P 37/08; A61K 31/07

(57) ABSTRACT

In one embodiment, the invention provides a composition that includes therapeutically effective amount of any unadulterated pollen listed in FIG. 1.

The invention is comprised of specific mass(es) of the unadulterated pollen composition of one or multiple pollens per daily dose, a range of mass of vitamin A, a range of mass of vitamin D, and a suspension buffer facilitating absorption of the therapeutic composition.

The duration of the therapeutically effective amount of unadulterated pollen will preferably be administered 0-52 weeks prior to exposure to the allergen and for a period of 6-8 weeks. It could also be administered as a maintenance treatment during the allergic exposure and for multiple years.

In some embodiments, the invention provides a method for treating allergies including administering an effective amount of a composition comprised of unadulterated pollen, vitamin A, vitamin D and a suspension buffer as shown in FIG. 1.

### CROSS-REFERENCE TO RELATED APPLICATIONS

This application claims the benefit of U.S. Provisional Application No. 62/522,256, filed on Jun. 20, 2017, the contents of which are incorporated by reference herein.

### FIELD OF THE INVENTION

The present invention relates to a method, composition and apparatus for treating allergies (immunotherapy) in an organism with an immune system, and, more particularly, to a method, composition and apparatus for the treatment of Type I Allergic Hypersensitivities by immune desensitization through the oral mucosal route, preferably sublingual (SLIT), with a composition comprised of unadulterated allergen in conjunction with vitamins A, D and a suspension buffer.

### BACKGROUND OF THE INVENTION

In general, allergies are a reaction of the immune system to a foreign substance. Human beings possessing allergies, generally have an immune system that reacts quickly to a substance in the environment, called an allergen. If the person's immune system does not respond to the foreign substance (i.e., the allergen), then the person is not allergic to that particular foreign substance. Allergies to the user can occur in specific times of the year, or can occur year round. The allergies include reactions caused by pollen, insects, insect bites or stings, animal hair, dust mites and other common substances in the environment. If the allergen is in the environment, such as air, surfaces, food or drink, and then contacts a human or an animal, the immune system of the human or animal responds to the allergen. The body responds in a variety of ways to the allergen.

In general, when the human/user or animal is exposed to an allergen, a series of events take place. The body of the human or animal produces a particular type of antibody, known as IgE (Immunoglobulin E) that has a binding specificity to the allergen. These antibodies associate with mast cells and release various inflammatory mediators, including histamine. Mast cells are found throughout the body, in the linings of the respiratory system, gastrointestinal and integumentary where allergens can enter the body. Once the allergen cross-links IgE antibodies bound to mast cells, they are triggered to release inflammatory compounds, particularly histamine, which causes the itchiness or runny nose associated with allergic reactions. The point of entry of the allergen into the body of the human or animal could determine the type of reaction that the immune system produces. If the allergen is in the air, the symptoms of the allergic reactions will include reactions in the eyes, nose and lungs. If the allergen enters through the gastrointestinal (GI) tract, the reactions will occur in the mouth, stomach and intestines. In some cases, there are systemic responses that occur regardless of the entry point of the allergen and such responses include hives, decreased blood pressure, anaphylactic shock, or loss of consciousness. The responses to allergens could vary from mild irritation to life-threatening.

Pharmaceutical agents are often prescribed to treat the symptoms and inhibit the secondary effects of mast cell histamine release, and include widely used agents such as antihistamines, inhaled steroids, nasal decongestants and mast cell stabilizers. Another approach to treatment of allergic reactions is immunotherapy, which is most frequently administered as injections, and is known as allergy shots or compositions. Allergy shots or compositions, as currently administered into the subcutaneous region (i.e. situated or applied under the skin) of the forearm is an effective method of long term relief from allergy symptoms. Recently, sublingual immunotherapy (SLIT) has been utilized to treat grass allergies.

Generally, an affected human being is administered an allergen extract composition of varying dosages for a defined period of time, until the body stops responding to the allergen. Many human beings, or animals affected with allergies do not undergo immunotherapy as per their diagnosed allergen, and further treat their allergies with general medications. Moreover, human beings would not consider respective anti-allergic treatment for their pets or farm animals.

Specifically, 10%-30% of the world population suffers from immediate-type hypersensitivity reactions mediated by IgE, which are termed as type I hypersensitivities. Type I hypersensitivities are caused primarily by plant and food allergens. Current treatments for type I hypersensitivities include predominantly anti-histamines, steroids and leukotriene inhibitors. Despite their proven efficacy, these medications are largely addressing the symptoms of type I hypersensitivities and not the actual cause of the allergic response. Furthermore, administration of anti-histamines causes drowsiness and sleepiness.

An alternative to anti-histamines, steroids and leukotriene inhibitors, is immune desensitization during which the allergen is introduced to the patient either subcutaneously or sublingually. In countries like U.S., subcutaneous immunotherapy (SCIT) has been the only FDA approved therapy for decades. Sublingual immunotherapy (SLIT) has only been recently approved in the U.S., despite decades of use in Europe. Furthermore, as of now SLIT immunotherapy is the only treatment performed for grass and dust mites allergies.

Sublingual (SLIT) immunotherapy involves extraction of an allergen from its native source (pollen) and suspension in liquid form or tablet formulation. The allergen liquid extract or tablet is then, introduced to the patient sublingually daily for an extended period of time. The rationale behind allergen extraction lies in the reasoning that it allows for a standardized amount of allergen to be introduced to the patient. However, given the susceptibility of allergenic proteins to denaturing and loss of epitopes through which immune tolerance can be achieved, it is not necessarily the optimal approach.

Certain vitamins have been shown to exert an immunomodulatory effect that can be employed in allergic reactions. Specifically, vitamin D supplementation has been shown to increase levels of regulatory T-cells, a key immune cell type capable of modulating the immune system. In addition, Retinoic Acid, a major metabolite of vitamin A, has also been shown to increase levels of regulatory T-cells. Given the nature of allergic reactions as aberrant immune responses, both vitamin A and vitamin D are suitable pharmacological tools that can be utilized in allergy treatments. Combining vitamins A and D with unadulterated allergen compositions delivered sublingually increases the effectiveness of sublingual immunotherapy. Mechanistically, both vitamin D and A, exert their immunomodulatory effect on T-regs. The increased activity of T-regs due to vitamin A and D, modulates the excessive response of the immune system towards the allergen.

Hence, there is long felt need for a pharmaceutical composition to treat the allergic reactions that is easier to administer, with less pain, and is effective for immunotherapy. Such composition should result in a high titer of polyclonal IgA antibodies capable of binding to the allergen, facilitating its removal and resulting in low production of IgE antibodies.

### Definitions

The term ‘oral mucosal’ refers to any surface in the oral cavity on which the therapeutic composition can be delivered and absorbed either in a suspension buffer or solid form. Sublingual (SLIT) is a type of oral mucosal delivery route.

The term SLIT refers to sublingual immunotherapy.

The term ‘sublingual’ refers to any surface beneath the tongue where the therapeutic composition can be absorbed.

### BRIEF DESCRIPTION OF FIGURES

FIG. 1 Table for synthesis of the therapeutic composition comprised of components listed in columns A and B according to one embodiment of the present invention.

FIG. 2 Ampule system for preparing and delivering the therapeutic composition according to one embodiment of the present invention.

FIG. 3 Ampoule matrix apparatus for therapeutic composition dosage to be delivered to the patient according to one embodiment of the present invention.

### DETAILED DESCRIPTION

The present invention discloses a pharmaceutical composition for treating Type I hypersensitivities by immune desensitization through sublingual (SLIT) immunotherapy and generally the oral mucosal route using unadulterated allergen, vitamin A, vitamin D and a suspension buffer. A pharmaceutical composition for treatment of allergies, comprises at least one of an unadulterated allergen, a suspension buffer, a dose of vitamin A, a dose of vitamin D and optionally one or more other pharmaceutically acceptable excipient(s). The said composition induces tolerance to a user for allergies caused by tree pollen, or any other type I allergies. The composition administered of one or more unadulterated allergens to at least one of a human/animals.

In one embodiment, the composition comprises one or more unadulterated allergen substance(s), which cause an allergic reaction in a human or animal.

In another embodiment, the composition used to deliver the allergen(s) is comprised of a suspension buffer, vitamin A and vitamin D at a range of doses. Specifically for vitamin A the dose range can vary daily from 0.001 μg to 3,000 μg. In the case of vitamin D, the dose range can vary daily from 1 I.U to 4,000 IU (1 I.U=5 μg cholecalciferol).

In some embodiments, the use of a suspension buffer provides an alternative to allergen extraction. The buffer can be mixed with unadulterated allergen shortly before sublingual administration. This approach offers several advantages compared to the existing allergen extraction practices for the listed reasons:
1. Minimizes degradation and denaturing of proteins found in pollen.
2. Produces a much wider array of pollen proteins.
3. Preserves epitopes on both allergenic and non-allergenic proteins allowing binding and production of patient immunoglobulins.
4. Allows for delivery of gradually increasing amounts of allergen to patient.
5. Allows for combination of allergens in one single suspension.
6. Increases patient compliance to therapy.
7. Does not require any special storage conditions of allergen.

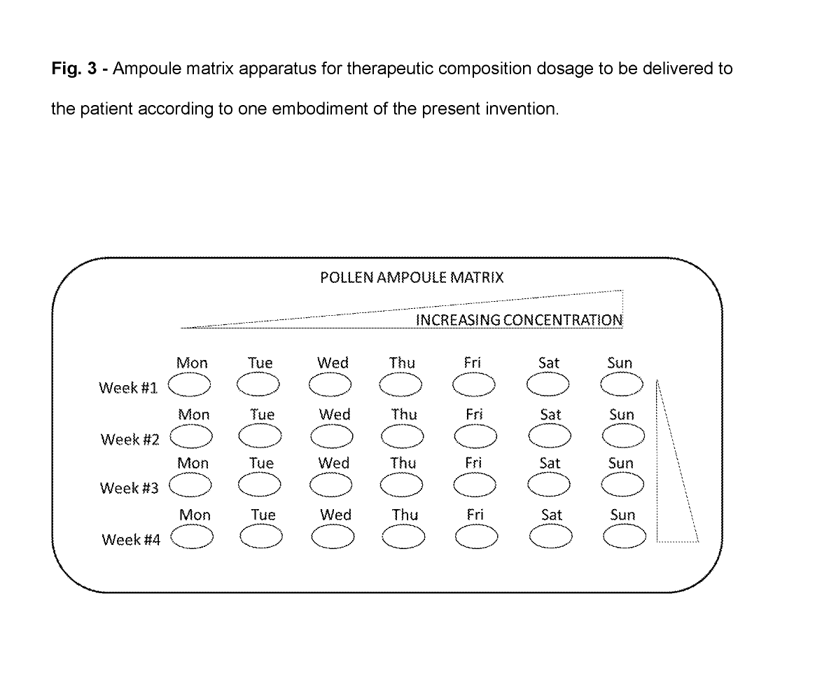

To reduce adverse side effects during sub-lingual immunotherapy (SLIT) or oral mucosal delivery, the allergen dosage can be increased gradually. Currently, patients undergoing SLIT do not have this choice (allergen extracts or tablets are typically at a pre-defined concentration). Using the pollen ampoule matrix apparatus (FIG. 3), dosage can be increased accordingly based on the patient's profile. Furthermore, use of the ampoule matrix apparatus increases patient compliance due to the defined daily/weekly architecture of the matrix. The ampoule matrix apparatus also allows for combination of allergens for patients who are allergic to more than one allergens. Currently, combination therapy requires use of multiple vials of allergen extracts and visits to a physician office daily or weekly.

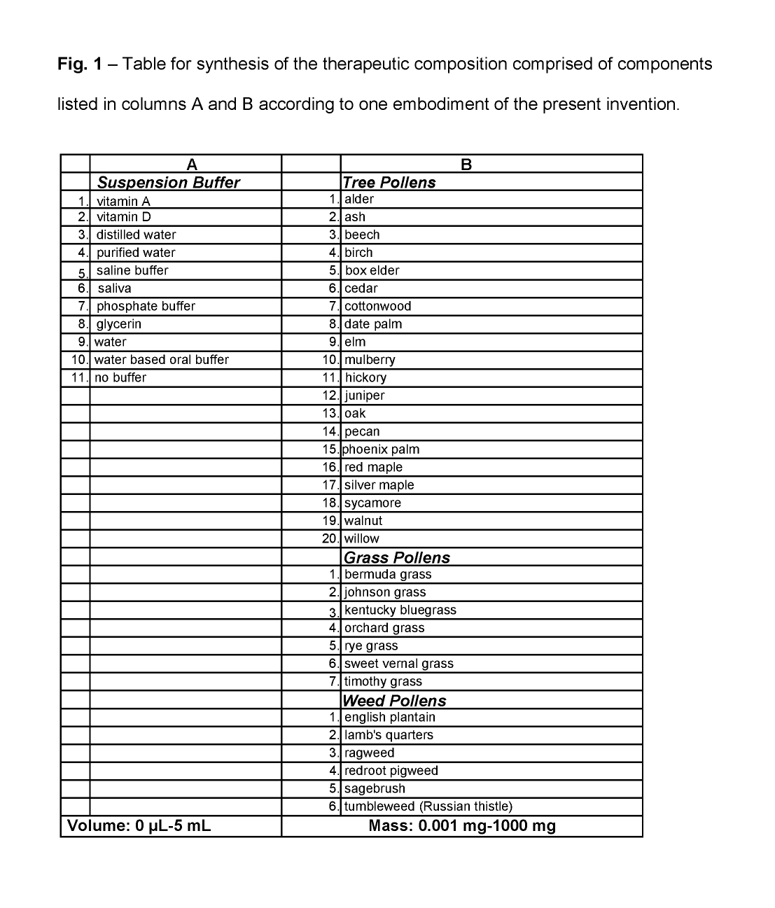

In another embodiment, the allergen composition of the ampoule matrix apparatus can be comprised of a total mass ranging from 0.001 mg to 1,000 mg. It can be increased incrementally on a daily basis throughout the duration of the treatment. The volume of the suspension buffer may remain constant in all ampoules and be comprised of any or none of the buffers listed in FIG. 1. In some embodiments, the suspension buffer could be omitted and vitamin A and D be directly mixed with the allergen. The composition can be directly delivered sublingually as powder form.

### SUMMARY OF DISCLOSURE

The invention provides methods for treating type I allergies with an inventive composition. The invention relates to the fields of biomedicine, immunology, allergy and molecular biology.

1. A method of treating type I allergic reactions, comprising the steps of administering via the route of oral mucosa including sublingually an allergic patient.

2. A therapeutic composition comprised of one or more of the unadulterated allergenic pollens shown in FIG. 1 in column B and a composition comprised of vitamin A, D and suitable delivery buffer listed but not limited to those in column A:

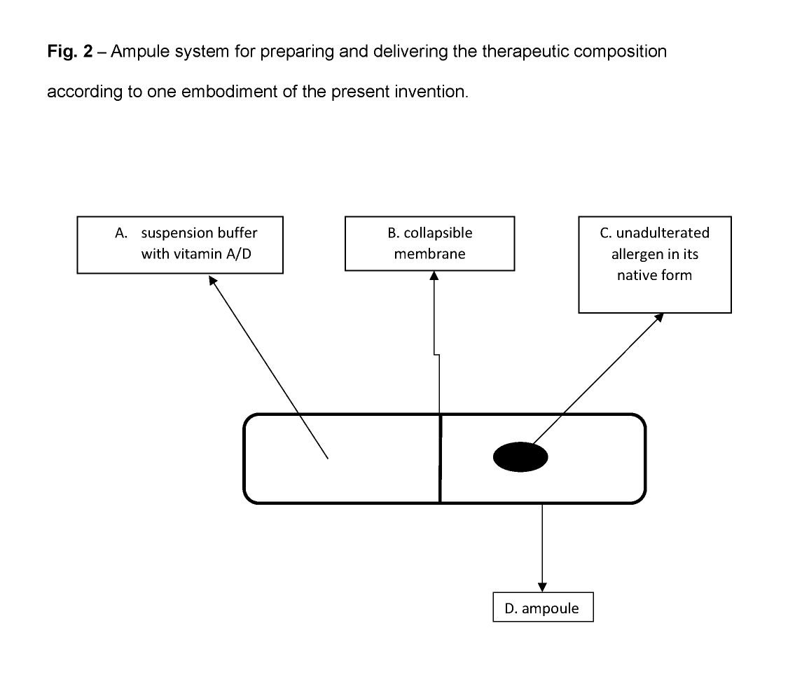

3. An apparatus shown in FIG. 2 for packaging, compartmentalizing and administering the therapeutic composition according to claim 2:

4. An apparatus shown in FIG. 3 for administering a variable, monthly dosage schedule of the therapeutic composition for treating type I allergies according to claim 2.

5. The composition according to claim 2, wherein the said is administered once and repeated for a period of time ranging from 1 to 120 days, preferably on a daily basis, once a year or if needed more than once a year.
